(19) United States
(12) Patent Application Publication
(10) Pub. No.: US 2020/0000713 A1
(43) Pub. Date: Jan. 2, 2020

(54) INJECTABLE POLYMER MICRO-DEPOTS FOR CONTROLLED LOCAL DRUG DELIVERY

(71) Applicant: Massachusetts Institute of Technology, Cambridge, MA (US)

(72) Inventors: Nitasha Bennett, Cambridge, MA (US); Darrell J. Irvine, Arlington, MA (US); Paula T. Hammond, Newton, MA (US)

(21) Appl. No.: 16/479,070

(22) PCT Filed: Jan. 19, 2018
(86) PCT No.: PCT/US2018/014449

§ 371 Date: Jul. 18, 2019

Related U.S. Application Data
(60) Provisional application No. 62/448,765, filed on Jan. 20, 2017.

(51) Int. Cl. A61K 9/00; A61K 9/16; A61K 49/00; A61K 47/38; A61K 9/06

(52) U.S. Cl. CPC A61K 9/0024; A61K 9/1658; A61K 9/06; A61K 9/1682; A61K 47/38; A61K 49/0091

(57) ABSTRACT

A pharmaceutical composition, comprising a particle (micro-depot) that includes silk fibroin and at least one active pharmaceutical ingredient (API). The particle can be suspended in carboxymethyl cellulose to form an injectable pharmaceutical composition.

### RELATED APPLICATIONS

This application claims the benefit of priority to U.S. Patent Application Ser. No. 62/448,765, filed Jan. 20, 2017, the contents of which are hereby incorporated by reference.

### BACKGROUND OF THE INVENTION

Controlled local drug delivery, for example within a local diseased/infected tissue or within tumors, is of great interest in many therapeutic applications. Localized bolus administration (e.g., via a syringe) does not ensure injected therapeutics remain in the local tissue, and often rapidly clear from the injection site (minimizing efficacy in the target tissue) while disseminating systemically (leading to undesirable side effects). Many drug delivery technologies have been pursued with this general goal, but often with a number of limitations such as low drug loads, low encapsulating efficiency, poor long-term stability, and specificity to the active pharmaceutical ingredients. A need exists for controlled delivery formulations suitable for delivery of therapeutic agents into a local tissue site.

### SUMMARY OF THE INVENTION

In certain embodiments, the invention relates to a particle comprising silk fibroin and at least one active pharmaceutical ingredient (API).

In certain embodiments, the invention relates to a method of manufacturing any of the particles described herein, comprising:
combining silk fibroin and at least one API in an aqueous solution;
placing the aqueous solution into a mold cavity, thereby coating a mold cavity with a silk/API layer;
placing a water-soluble polymer into the mold cavity, thereby coating the mold cavity with a silk/API/polymer layer;
removing the silk/API/polymer layer from the mold cavity; and
dissolving the polymer, thereby forming the particle.

In certain embodiments, the invention relates to a pharmaceutical composition, comprising:
a carboxymethyl cellulose (CMC) gel comprising CMC and a pharmaceutically acceptable carrier; and
a plurality of particles suspended within the CMC gel, wherein at least one particle is any of the particles described herein.

In certain embodiments, the invention relates to a method of manufacturing any of the pharmaceutical compositions described herein, comprising:
combining silk fibroin and at least one API in an aqueous solution;
placing droplets of the aqueous solution onto a surface, thereby forming silk/API droplets;
annealing the silk/API droplets, thereby forming a plurality of particles;
coating the particles with CMC, thereby forming a silk/API-loaded film; and
hydrating the silk/API-loaded film, thereby forming the pharmaceutical composition.

In certain embodiments, the invention relates to a method of treating cancer comprising administering by intratumoral injection to a cancerous tumor in a subject in need thereof a therapeutically effective amount of any of the pharmaceutical compositions described herein, provided the API is not an imaging agent.

In certain embodiments, the invention relates to a method of imaging a cancerous tumor comprising administering by intratumoral injection to the cancerous tumor in a subject in need thereof an effective amount of any of the pharmaceutical compositions described herein, provided the API comprises an imaging agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram illustrating the process of fabrication of silk micro-depots for sustained drug delivery to diseased organs.

FIG. 2A is a scatter plot showing the amount of recovered API after a degradation of the silk micro-depots (in % of initial load).

FIG. 2B is a microphotograph of micro-depots (top: single layer; bottom: double layer) formed in the wells of the silicon array.

FIG. 2C is a bar plot showing the amounts of recovered API (in micrograms) as a function of the number of layers of the micro-depots. Layered silk deposition allows for increased drug loading.

FIG. 2D is a schematic diagram illustrating the effect of methanol annealing on the structure of silk fibroin. Increased β-sheet content slows release of API from silk tips.

FIG. 2E is a plot showing the amount of API released from silk micro-depots as a function of time and the number of layers.

FIG. 3A is a schematic diagram illustrating the method of fabrication of an injectable composition comprising the silk micro-depots. The photograph shows a 6% loading of silk tips in carboxymethylcellulose (CMC) gel.

FIG. 3B is a photograph demonstrating the appearance of the injectable composition. Shear-thinning properties allow dispersing of silk micro-depots with a syringe.

FIG. 3C is a scatter plot showing the amount of API recovered from aliquots dispensed under the indicated conditions.

FIG. 3D is a fluorescence microscopy image showing the distribution of silk micro-depots in the tumor tissue.

FIG. 4A is a scatter plot of recovered AF647 dye as a function of the number of silk layers as percent of total loaded.

FIG. 4B is a scatter plot of the amount of recovered AF647 dye (nmol) as a function of the number of silk layers.

FIG. 5A is a bar plot showing the amounts of recovered API (as a percentage of the total) as a function of the number of layers of the micro-depots.

FIG. 5B is a plot showing the amount of API released from silk micro-depots as a function of time and the number of layers.

FIG. 6 is a schematic representation of therapeutically altering a tumor microenvironment.

FIG. 7A is a schematic representation of APIs for pilot in vivo studies.

FIG. 7B is a timeline showing the study protocol with STING agonist delivery in neoadjuvant for administration of IL2-albumin fusion (MSA-IL2), anti-PD-1 antibody (αPD-1 or a-PD-1), and STING agonist (CDN).

FIG. 7C is a fluorescence image showing the silk and gel after injection into a primary 4T1 tumor.

FIG. 7D shows fluorescence images of tumors in untreated (left) and CDN+MSA-IL2+αPD-1-treated (right) mice.

FIG. 7E is a graph showing tumor area as a function of time in untreated, CDN-treated, MSA-IL2+a-PD-1-treated, and CDN+MSA-IL2+a-PD-1-treated subjects.

FIG. 7F is a graph of percent survival as a function of time in untreated, CDN-treated, MSA-IL2+a-PD-1-treated, and CDN+MSA-IL2+a-PD-1-treated subjects.

FIG. 8A is a schematic representation of representative protein cargo and representative small molecule cargo that may be loaded onto soluble silk fibroin. Drug loading is performed by mixing of aqueous solutions of drug and silk fibroin.

FIG. 8B is a schematic representation of a representative preparation of the silk/drug matrix. The first step shows fluorescent silk/drug droplets imaged by confocal microscopy on a PDMS surface. The second step shows a cellulose film applied to the silk/drug droplets on the PDMS surface. The third step shows removal of the cellulose film with the silk/drug droplets.

FIG. 8C is a series of photographs of (1) a cellulose film embedded with silk/drug matrix, (2) cellulose/silk/drug after hydration, and (3) a syringe loaded with hydrated cellulose/silk/drug.

FIG. 9A is a schematic representation of increasing the β-sheet content of silk. B-sheet content is increased by annealing, such as dehydrating a silk film via exposure to methanol vapor (i.e., methanol annealing) or extended heating at increased humidity (i.e., water annealing).

FIG. 9B is a graph of release of IgG (%) from a silk matrix as a function of time for matrices subjected to water annealing at various temperatures. Water annealing is effective for slow release of protein cargo and maintenance of protein stability.

FIG. 9C is a scatter plot of antigen binding (as measured by ELISA) of IgG released from a silk matrix. The matrices were subjected to water annealing at various temperatures. IgG retains its binding capacity when annealed at a temperature of about 60° C. or lower.

FIG. 10A is a graph of recovery of ivermectin (IVM) (%) from a silk matrix as a function of time for a matrix annealed via methanol dehydration and for a control matrix not subjected to annealing. Methanol annealing is effective for slow release of the small molecule IVM.

FIG. 10B is a scatter plot of total IVM recovery for a silk matrix subjected to methanol annealing (closed diamonds) and a silk matrix not subjected to an annealing process (open diamonds). The recovery of IVM from the annealed matrix is equivalent to the recovery of IVM from the control.

FIG. 11A is a fluorescence confocal microscope image of a silk matrix loaded with IgG and suspended in a cellulose gel. The silk (grey) was not subjected to annealing.

FIG. 11B is a fluorescence confocal microscope image of a silk matrix loaded with IgG and suspended in a cellulose gel. The silk (grey) was subjected to methanol annealing.

FIG. 12A is a fluorescence confocal microscope image of a mouse 4T1 tumor (tumor boundary marked with dashed lined) immediately following injection of a silk matrix suspended in a cellulose gel. The silk (grey) was not subjected to annealing.

FIG. 12B is a fluorescence confocal microscope image of a mouse 4T1 tumor (tumor boundary marked with dashed lined) immediately following injection of a silk matrix suspended in a cellulose gel. The silk (grey) was subjected to methanol annealing.

FIG. 13A is a series of fluorescent IVIS images showing the retention in mouse tumors of fluorescent silk in cellulose gels. Different molecular weight cellulose, and different cellulose concentrations were investigated.

FIG. 13B is a photograph of silk loaded onto different concentrations of cellulose. Increased cellulose concentration resulted in thicker gels.

FIG. 13C is a scatter plot showing radiance efficiency of silk depots (also called “silk implants” once injected) in cellulose at differing cellulose concentrations or differing cellulose molecular weights.

FIG. 13D is a graph of radiance efficiency as a function of time of silk depots in cellulose at differing cellulose concentrations or differing cellulose molecular weights. Silk depots were retained better when suspended in less viscous cellulose gels.

FIG. 14A a timeline showing the study protocol for 4T1 tumors treated with an αCD137 antibody delivered as a soluble (sol) injection (four doses) or via silk depots (two doses) in combination with systemic IL-2 (I) and anti-PD1 (P). Tumor growth was measured over time.

FIG. 14B is a graph of total radiant efficiency as a function of time for sol αCD137 antibody or αCD137 antibody delivered via a silk depots. Delivery of αCD137 by silk depots results in longer retention in the tumor.

FIG. 14C is a graph of tumor area (mm²) as a function of time (d) after injection with sol αCD137 (grey squares), αCD137 in silk depots (open triangles), or untreated (black diamonds). The extended retention of αCD137 when delivered via silk depots enables a similar reduction in tumor growth to as compared to sol injection, but requires less frequent dosing.

### DETAILED DESCRIPTION OF THE INVENTION

### Overview

A description of example embodiments of the invention follows.

Disclosed herein are polymer micro-depots that use silk protein as a matrix and that can be employed in injectable pharmaceutical formulations. These micro-depots are molded solid silk matrices entrapping biologic or small molecule drugs, fabricated under aqueous conditions. Silk preserves the structure and bioactivity of entrapped macromolecules, enabling release of proteins with fully intact 3D folded structures. Control of silk β-sheet crystallinity allows release rates from the silk matrix to be tailored to specific applications. Variation of drug/silk ratios during molding of the micro-depots enables any desired drug loading to be achieved. The encapsulation efficiency can be very high, because a molding process is used where no added material is lost during the fabrication process. The molding process could be adapted to a variety of additional biodegradable materials.

The pharmaceutical compositions disclosed herein possess a number of advantages over the existing technologies, such as high drug loading, high drug encapsulation efficiency, long-term stability of the pharmaceutically active load, and ability to be employed with diverse classes of active pharmaceutical ingredients.

In an example embodiment, the present invention is a pharmaceutical composition, comprising a particle. The particle comprises silk fibroin; and at least one active pharmaceutical ingredient (API).

In another example embodiment, the present invention is a pharmaceutical composition, comprising a carboxymethyl cellulose (CMC) gel and a plurality of particles suspended within the CMC gel, wherein at least one particle comprises silk fibroin and at least one API.

In another example embodiment, the present invention is a method of manufacturing a pharmaceutical composition. The method comprises combining silk fibroin and at least one API in an aqueous solution; placing the aqueous solution into a mold cavity, thereby coating a mold cavity with a silk/API layer; placing a water soluble polymer into the mold cavity, thereby coating the mold cavity with a silk/API/polymer layer; removing the silk/API/polymer layer from the mold cavity; and dissolving the polymer, thereby forming a silk/API particle. In various aspects of the method, the silk/API layer can be exposed to methanol vapor. In other aspects, the silk/API particle can be combined with a carboxymethyl cellulose (CMC).

The teachings of all patents, published applications and references cited herein are incorporated by reference in their entirety.

### Silk Fibroin

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers. Silk is naturally produced by various species, including, without limitation: Antheraea mylitta; Antheraea pernyi; Antheraea yamamai; Galleria mellonella; Bombyx mori; Bombyx mandarins; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

Silk fibroin proteins offer desirable material characteristics for a number of applications that take advantage of the nature of biological materials, such as biocompatibility. Silk fibroin of the Bombyx mori silkworm has come of considerable interest in this context, owing to its attractive mechanical (B. D. Lawrence, et al., Journal of Materials Science 2008, 43, 6967-6985; S. Sofia et al., Journal of Biomedical Materials Research 2001, 54, 139-48; L. Meinel et al., Bone 2006, 39, 922-31; H.-J. Jin et al., Biomacromolecules 2002, 3, 1233-9), biological (M. Santin et al., Journal of Biomedical Materials Research 1999, 46, 382-9; E. M. Pritchard et al., Journal of Controlled Release: Official Journal of the Controlled Release Society 2010, 144, 159-67), and optical properties (H. Perry et al., Advanced Materials 2008, 20, 3070-3072; B. D. Lawrence et al., Biomacromolecules 2008, 9, 1214-20) for use in biomedical, optical, electro-optical, industrial and other applications.

According to various embodiments, silk fibroin may comprise any of a variety of silk fibroin proteins including, but not limited to, those described herein and in WO 97/08315 and U.S. Pat. No. 5,245,012.

In certain embodiments, silk fibroin is B. mori silk fibroin, which consists of a light chain (M_{w} approximately 26 kDa) and a heavy chain (M_{w} approximately 390 kDa) linked by a disulfide bond. Silk fibroin is a block copolymer rich in hydrophobic β-sheet forming blocks linked by small hydrophilic linker segments or spacers. The crystalline regions are primarily composed of glycine-X repeats, where X is alanine, serine, threonine, or valine. Within these domains lie subdomains rich in glycine, alanine, serine, and tyrosine. The result is a hydrophobic protein that self-assembles to form strong and resilient materials. The dominance of the β-sheet-forming regimes within the fibroin structure impart the protein-based materials with high mechanical strength and toughness. In certain embodiments, the silk fibroin is from about 35% to about 65% beta-sheet crystalline, for example, from about 40% to about 55% beta-sheet crystalline after water annealing, or from about 50% to about 60% beta-sheet crystalline after methanol annealing.

Silk protein solutions can be prepared by any conventional methods known to one skilled in the art. A brief exemplary process for preparing a silk protein solution is provided in order to provide a better understanding of some of the principles of the present invention. In some embodiments, B. mori cocoons are boiled for about 30 minutes in an aqueous solution (e.g. 0.02 M Na₂CO₃). The cocoons are then rinsed, for example, with water to extract the sericin proteins and the extracted silk is dissolved in an aqueous salt solution. Salts useful for this purpose include, lithium bromide, lithium thiocyanate, calcium nitrate or other chemical capable of solubilizing silk. In some embodiments, a strong acid such as formic or hydrochloric may also be used. In some embodiments, the extracted silk is dissolved in about 9-12 M LiBr solution. Regardless of the specific extraction method(s) used, the salt is consequently removed using, for example, dialysis.

In some embodiments, a silk protein solution may be substantially free of sericin. As used herein, “substantially free of sericin” means that sericin is absent from such a preparation, or present in such a trace amount that it does not affect the subsequent step or steps of silk fibroin processing or its downstream application. In some embodiments, a trace amount of sericin that may be present in a silk fibroin preparation is present in concentrations less than about 0.5%, less than about 0.4%, less than about 0.3%, less than about 0.2%, less than about 0.1%, less than about 0.05%, less than about 0.04%, less than about 0.03%, less than about 0.02%, less than about 0.01%, or lower. In some embodiments, a trace amount of sericin that may be present in a silk fibroin preparation is present in a concentration that is below a detectable threshold by conventional assays used in the art.

In some embodiments, one or more biocompatible polymers are added to a silk protein solution in order to form a pharmaceutical composition described herein. Suitable biocompatible polymers compatible with various embodiments of the present invention include, but are not limited to, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), polyanhydrides (U.S. Pat. No. 5,270,419), poly(vinyl pyrrolidone), and other biocompatible polymers. In some embodiments, the PEO has a molecular weight from, 400,000 to 2,000,000 g/mol. In some embodiments, the molecular weight of the PEO is about 900,000 g/mol. As contemplated by the present invention, two or more biocompatible polymers can be directly added to the aqueous solution simultaneously or sequentially.

In some embodiments, a silk solution and/or aqueous solution comprising silk protein has a concentration of about 0.1 to about 30 weight percent of silk protein. In some embodiments, the silk solution and/or aqueous solution comprising silk protein has a concentration of about 1 to about 20 weight percent of silk protein. In some embodiments, the silk solution and/or aqueous solution comprising silk protein has a concentration of about 1 to about 10 weight percent of silk protein. In some embodiments, the silk solution and/or aqueous solution comprising silk protein has a concentration of about 1 to about 5 weight percent of silk protein. In some embodiments, the silk solution and/or aqueous solution comprising silk protein has a concentration of about 5 to about 10 weight percent of silk protein.

### Glossary

The term “nucleic acid,” or “polynucleotide,” means a biopolymer composed of nucleotides. As used herein, dinucleotides are included in the term “nucleic acid”

The term “small molecule” means a low molecular weight (<1 kDa) organic compound.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. An example of a protein is an antibody.

An “imaging agent” is a bioluminescent or chemiluminescent label. Such labels include small molecules or polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof.

An imaging agent may also be a paramagnetic compound, such as a metal. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(II), a Mn(II), or an element comprising a Fe element. Paramagnetic compounds can also comprise a neodymium iron oxide (NdFeO₃) or a dysprosium iron oxide (DyFeO₃). Examples of elements that are useful in magnetic resonance imaging include gadolinium, terbium, tin, iron, or isotopes thereof (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey at al., (1984) Physiol. Chem. Phys. Med. NMR 16, 145-155; Runge et al., (1984) Invest. Radiol. 19, 408-415 for discussions on in vivo nuclear magnetic resonance imaging.)

“STING” is an abbreviation of “stimulator of interferon genes”, which is also known as “endoplasmic reticulum interferon stimulator (EMS)”, “mediator of IRF3 activation (MITA)”, “MPYS” or “transmembrane protein 173 (TM173)”. STING is a transmembrane receptor protein and is encoded by the gene TMEM173 in human. In response to viral infection, STING activates STATE (signal transducer and activator of transcription 6) to induce (Th2-type), increase (IL-12) or decrease (IL-10) production of various cytokines, including the chemokines CCL2, CCL20, and CCL26 (Chen et al., 2011).

The term “STING agonist,” as used herein, refers to a substance that activates the receptor STING in vitro or in vivo. According to the invention, a compound is deemed to be a STING agonist if: it induces Type I interferons in vitro in human or animal cells that contain STING; and it does not induce Type I interferons in vitro in human or animal cells that do not contain STING. A typical test to ascertain whether a ligand is a STING agonist is to incubate the ligand in a wild-type human or animal cell line and in the corresponding cell line in which the STING coding gene has been genetically inactivated by a few bases or a longer deletion (e.g. a homozygous STING knockout cell line). An agonist of STING will induce Type I interferon in the wild-type cells but will not induce Type I interferon in the cells in which STING is inactivated. Some cyclic dinucleotides are STING agonists.

Representative examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP).

Human CD73 (also referred to as 5′-nucleotidase, ecto; NT5E; or 5NT) is a 574 amino acid residue protein (Accession No. AAH6593). Eukaryotic CD73 functions as a noncovalent homodimer with two structural domains, wherein the N- and C-terminal domains are connected by a hinge region that enables the enzyme to undergo large domain movements and switch between open and closed conformations (Knapp, K. et al. (2012) Structure 20:2161-73).

As used herein, the terms “CD73 inhibitor”, “CD73 blocker”, “adenosine by 5′-nucleotidase, ecto inhibitor”, “NT5E inhibitor”, “5NT inhibitor” and all other related art-accepted terms refer to a compound capable of modulating, either directly or indirectly, the CD73 receptor in an in vitro assay, an in vivo model, and/or other means indicative of therapeutic efficacy. The terms also refer to compounds that exhibit at least some therapeutic benefit in a human subject. An CD73 inhibitor may be a competitive, noncompetitive, or irreversible CD73 inhibitor. “A competitive CD73 inhibitor” is a compound that reversibly inhibits CD73 enzyme activity at the catalytic site; “a noncompetitive CD73 inhibitor” is a compound that reversibly inhibits CD73 enzyme activity at a non-catalytic site; and “an irreversible CD73 inhibitor” is a compound that irreversibly eliminates CD73 enzyme activity by forming a covalent bond (or other stable means of inhibiting enzyme function) with the enzyme.

CD73 inhibitors can modulate purinergic signaling, a type of extracellular signaling mediated by purine nucleotides and nucleosides such as ATP and adenosine. Purinergic signaling involves the activation of purinergic receptors in the cell and/or in nearby cells, resulting in the regulation of cellular functions. The enzymatic activity of CD73 plays a strategic role in calibrating the duration, magnitude, and chemical nature of purinergic signals delivered to various cells (e.g., immune cells). Alteration of these enzymatic activities can change the course or dictate the outcome of several pathophysiological events, including cancer, autoimmune and inflammatory diseases, infections, atherosclerosis, and ischemia-reperfusion injury, suggesting that these ecto-enzymes represent novel therapeutic targets for managing a variety of disorders.

Studies using tissues that overexpress CD73 and using CD73 knock-out mice have provided evidence that CD73 inhibitors have potential utility for melanomas, lung cancer, prostate cancer, and breast cancer (see, e.g., Sadej R. (2006) Melanoma Res 16:213-22). Because higher expression levels of CD73 are associated with tumor neovascularization, invasiveness, resistance to chemotherapy, and metastasis, CD73 inhibitors can be used to control tumor progression and metastasis.

Examples of CD73 inhibitors include, but are not limited to, α,β-methylene ADP (APCP) and anti-mouse CD73 mAb clone TY/23.

Carcinoma-associated fibroblasts (CAFs) are key players in the multicellular, stromal-dependent alterations that contribute to malignant initiation and progression. Indeed, interactions of CAFs with cellular components of the immune system contribute, to a large extent, to the tumor-promoting role of CAFs through immunosuppression and sustained inflammation. Moreover, the CAF status has an impact on the clinical behavior of a tumor, in particular early and targeted metastasis. Fibroblast-directed therapy can be envisioned as either “ablating” CAFs by interfering with their survival or “normalizing” them by interfering with secreted protumorigenic signals.

CAF modulators include, but are not limited to β-aminopropionitrile, losartan potassium, 4-methylumbelliferone, ruxolitinib, and dasatinib.

Immunomodulatory antibodies are antibodies used to induce, enhance, or suppress an immune response. Immunomodulatory treatment regimens often have fewer side effects than existing drugs, including less potential for creating resistance when treating microbial disease. Examples of immunomodulatory antibodies include, but are not limited to, anti-CD137 (4-1BB), anti-CD40, anti-CD134 (OX40), anti-CD152 (CTLA-4), anti-cd274 (PD-L1), anti-CD279 (PD-1), and anti-CD366 (Tim3) antibodies.

An “adjuvant” is a pharmacological or immunological agent that modifies the effect of other active pharmaceutical ingredients. Examples of adjuvants include, but are not limited to, CpG oligonucleuotides, monophosphoryl lipid A, and Pam3Cys.

As used herein, a “cytokine” is a small protein (e.g., less than 20 kDa) that is important in cell signalling. Cytokines include, but are not limited to, chemokines, interleukins, lymphokines, and tumor necrosis factors. Examples of cytokines, including chemokines, include, but are not limited to, CCL5, CXCL10, CCL21, CXCL13, CXCL12, GM-CSF, interleukin-1, interleukin-15, and interleukin-12 (which can be recombinant with or without Fc domain).

As used herein, the term “water-soluble polymer” is a polymer generally having a solubility of at least 10 g/l in water at a temperature between 20° C. and 90° C. and at a pH between 3 and 12, in particular at a pH between 3 and 9. Examples of water-soluble polymers include: polyacrylic acid, gelatin, hydrolyzed gelatin, sodium polyacrylate, partially neutralized polyacrylic acid, polyacrylic acid-starch complexes, polyvinyl alcohol, polyvinylpyrrolidone, hydroxypropylcellulose, hydroxypropyl methylcellulose, hydroxyethyl cellulose, methylcellulose, carmellose sodium, carboxyvinyl polymer, methoxy ethylene-maleic anhydride copolymers, N-vinyl acetamide copolymers, xanthan gum, and gum arabic.

As used herein, the term “pharmaceutically acceptable carrier” refers to a pharmaceutically-acceptable material, composition or vehicle for administration of an active agent described herein. Pharmaceutically acceptable carriers include any and all solvents, dispersion media, coatings, antibacterial and antifungal agents, isotonic and absorption delaying agents, and the like which are compatible with the activity of the active agent and are physiologically acceptable to the subject. Some examples of materials which can serve as pharmaceutically-acceptable carriers include: (i) sugars, such as lactose, glucose and sucrose; (ii) starches, such as com starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, com oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C₂-C₁₂ alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation. For formulations described herein to be administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while com starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutically acceptable carriers can vary in a formulation described herein, depending on the administration route. The formulations described herein can be delivered via any administration mode known to a skilled practitioner. For example, the formulations described herein can be delivered in a systemic manner, via administration routes such as, but not limited to, oral, and parenteral including intravenous, intramuscular, intraperitoneal, intradermal, and subcutaneous. In some embodiments, the formulations described herein are in a form that is suitable for injection. In other embodiments, the formulations described herein are formulated for oral administration.

When administering parenterally, a formulation described herein can be generally formulated in a unit dosage injectable form (solution, suspension, emulsion). The formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g., PBS).

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to formulations described herein, however, any vehicle, diluent, or additive used should have to be biocompatible with the active agents described herein. Those skilled in the art will recognize that the components of the formulations should be selected to be biocompatible with respect to the active agent. This will present no problem to those skilled in chemical and pharmaceutical principles, or problems can be readily avoided by reference to standard texts or by simple experiments (not involving undue experimentation).

For in vivo administration, the formulations described herein can be administered with a delivery device, e.g., a syringe. Accordingly, an additional aspect described herein provides for delivery devices comprising at least one chamber with an outlet, wherein the at least one chamber comprises a pre-determined amount of any formulation described herein and the outlet provides an exit for the formulation enclosed inside the chamber. In some embodiments, a delivery device described herein can further comprise an actuator to control release of the formulation through the outlet. Such delivery device can be any device to facilitate the administration of any formulation described herein to a subject, e.g., a syringe, a dry powder injector, a nasal spray, a nebulizer, or an implant such as a microchip, e.g., for sustained-release or controlled release of any formulation described herein.

As used herein, the term “characteristic size” means characteristic diameter, or, for a plurality of particles, mean, median, or mode diameter. In some embodiments, “characteristic size” for a plurality of particles means that at least about 50%, at least about 60%, at least about 70%, at least about 80%, or at least about 90% of the particles have the recited characteristic size.

As used herein, the phrase “targeting agent” means any moiety a moiety that localizes to or away from a specific locale. The attachment of a targeting moiety to a compound increases the concentration of the compound at a site of treatment, for example, a tumor site. A targeting agent includes, but is not limited to, a lectin, glycoprotein, lipid or protein, e.g., an antibody, that binds to a specified cell type such as a tumor cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ as determined in an in vitro enzyme assay or cell culture (i.e., the concentration of the anticancer agent that achieves half-maximal inhibition of an enzyme or half-maximal inhibition of symptoms). Levels in plasma can be measured, for example, by high performance liquid chromatography. The effects of any particular dosage can be monitored by a suitable bioassay. The dosage can be determined by a physician and adjusted, as necessary, to suit observed effects of the treatment.

### Particles, Compositions, and Methods

In certain embodiments, the invention relates to a particle comprising: silk fibroin; and at least one active pharmaceutical ingredient (API).

In certain embodiments, the invention relates to any of the particles described herein, wherein the silk fibroin is Bombyx mori silk fibroin.

In certain embodiments, the invention relates to any of the particles described herein, wherein the API is a protein, a nucleic acid, or a small molecule.

In certain embodiments, the invention relates to any of the particles described herein, wherein the API is selected from: a STING agonist, a CD73 inhibitor, a CAF modulator, an immunomodulatory antibody, an adjuvant, a cytokine, and an imaging agent.

In certain embodiments, the invention relates to any of the particles described herein, wherein the API is selected from: an anti-CD73 antibody, IgG, ovalbumin, a HIV-1 envelope trimer protein, polylC, cyclic diguanylate monophosphate (CDN), Pam3CSK4, and ivermectin.

In certain embodiments, the invention relates to any of the particles described herein, wherein the particle has a characteristic size from about 50 μm to about 800 μm, for example, from about 100 μm to about 600 μm.

In certain embodiments, the invention relates to any of the particles described herein, wherein the API is suspended in the silk fibroin.

In certain embodiments, the invention relates to any of the particles described herein, wherein the amount of the API in the particle is from about 10 pg to about 0.6 μg, for example, from about 10 pg to about 5 ng. In certain embodiments, the API is a protein, and the amount of the API in the particle is from about 10 pg to about 2 ng.

In certain embodiments, the invention relates to any of the particles described herein, wherein the amount of silk fibroin in the particle is from about 50 pg to about 10 μg, for example, from about 50 pg to about 20 ng.

In certain embodiments, the invention relates to any of the particles described herein, wherein the mass ratio of silk fibroin to API is from about 100:1 to about 1:5. In certain embodiments, the API is a small molecule, and the mass ratio of silk fibroin to API is from about 100:1 to about 5:1.

In certain embodiments, the invention relates to any of the particles described herein, wherein the particle is substantially conical in shape.

In certain embodiments, the invention relates to any of the particles described herein, wherein the particle is substantially spherical.

In certain embodiments, the invention relates to a pharmaceutical composition, comprising: a carboxymethyl cellulose (CMC) gel comprising CMC and a pharmaceutically acceptable carrier; and a plurality of particles suspended within the CMC gel, wherein at least one particle is a particle described herein.

In certain embodiments, the invention relates to any of the pharmaceutical compositions described herein, wherein the pharmaceutically acceptable carrier is water.

In certain embodiments, the invention relates to any of the pharmaceutical compositions described herein, wherein the molecular weight of the CMC is from about 50 kDa to about 500 kDa. In certain embodiments, the invention relates to any of the pharmaceutical compositions described herein, wherein the molecular weight of the CMC is from about 80 kDa to about 300 kDa. In certain embodiments, the invention relates to any of the pharmaceutical compositions described herein, wherein the molecular weight of the CMC is about 90 kDa. In certain embodiments, the invention relates to any of the pharmaceutical compositions described herein, wherein the molecular weight of the CMC is about 250 kDa.

In certain embodiments, the invention relates to any of the pharmaceutical compositions described herein, wherein the concentration of CMC in the CMC gel is from about 1% to about 8% by weight. In certain embodiments, the invention relates to any of the pharmaceutical compositions described herein, wherein the molecular weight of the CMC is about 90 kDa; and the concentration of CMC in the gel is from about 4% to about 8% by weight. In certain embodiments, the invention relates to any of the pharmaceutical compositions described herein, wherein the molecular weight of the CMC is about 250 kDa; and the concentration of CMC in the gel is from about 1% to about 4% by weight.

In certain embodiments, the invention relates to any of the pharmaceutical compositions described herein, wherein the particles are present at a concentration from about 2 mg/mL to about 20 mg/mL in the CMC gel, for example, from about 3 mg/mL to about 15 mg/mL in the CMC gel.

In certain embodiments, the invention relates to a method of manufacturing any of the particles described herein, comprising: combining silk fibroin and at least one API in an aqueous solution; placing the aqueous solution into a mold cavity, thereby coating a mold cavity with a silk/API layer; placing a water-soluble polymer into the mold cavity, thereby coating the mold cavity with a silk/API/polymer layer; removing the silk/API/polymer layer from the mold cavity; and dissolving the polymer, thereby forming the particle.

In certain embodiments, the invention relates to any of the methods described herein, wherein the concentration of silk fibroin in the aqueous solution is from about 5 mg/mL to about 100 mg/mL.

In certain embodiments, the invention relates to any of the methods described herein, wherein the concentration of API in the aqueous solution is from about 1 mg/mL to about 25 mg/mL. In certain embodiments, the API is a protein, and the concentration of API in the aqueous solution is from about 1 mg/mL to about 10 mg/mL.

In certain embodiments, the invention relates to any of the methods described herein, further including annealing the silk/API layer.

In certain embodiments, the invention relates to any of the methods described herein, wherein annealing the silk/API layer comprises exposing the silk/API layer to methanol vapor at a temperature from about 20° C. to about 24° C. for a time period of about 9 hours (h) to about 48 h. In certain embodiments, the invention relates to any of the methods described herein, wherein the silk/API layer is exposed to methanol vapor for a period of from about 12 h to about 36 h. In certain embodiments, the invention relates to any of the methods described herein, wherein the silk/API layer is exposed to methanol vapor for a period of from about 18 h to about 24 h.

In certain embodiments, the invention relates to any of the methods described herein, wherein annealing the silk/API layer comprises heating, in the presence of a water bath, the silk/API layer at a temperature from about 30° C. to about 70° C. at a pressure from about 10 mmHg to about 30 mmHg for a time period from about 6 h to about 36 h. In certain embodiments, the pressure is about 20 mmHg. In certain embodiments, the period of time is from about 12 h to about 24 h.

In certain embodiments, the invention relates to any of the methods described herein, further comprising placing a second amount of the aqueous solution into the mold cavity, thereby coating the silk/API layer with a second silk/API layer.

In certain embodiments, the invention relates to any of the methods described herein, wherein the water-soluble polymer is selected from polyacrylic acid, gelatin, hydrolyzed gelatin, sodium polyacrylate, partially neutralized polyacrylic acid, polyacrylic acid-starch complexes, polyvinyl alcohol, polyvinylpyrrolidone, hydroxypropylcellulose, hydroxypropyl methylcellulose, hydroxyethyl cellulose, methylcellulose, carmellose sodium, carboxyvinyl polymer, methoxy ethylene-maleic anhydride copolymers, N-vinyl acetamide copolymers, xanthan gum, and gum arabic.

In certain embodiments, the invention relates to any of the methods described herein, further including combining the particle with a carboxymethyl cellulose (CMC).

In certain embodiments, the invention relates to a method of manufacturing any of the pharmaceutical compositions described herein, comprising: combining silk fibroin and at least one API in an aqueous solution; placing droplets of the aqueous solution onto a surface, thereby forming silk/API droplets; annealing the silk/API droplets, thereby forming a plurality of particles; coating the particles with CMC, thereby forming a silk/API-loaded film; and hydrating the silk/API-loaded film, thereby forming the pharmaceutical composition.

In certain embodiments, the invention relates to any of the methods described herein, further comprising removing the silk/API-loaded film from the surface prior to hydration.

In certain embodiments, the invention relates to any of the methods described herein, wherein the surface is polydimethylsiloxane (PDMS).

In certain embodiments, the invention relates to any of the methods described herein, wherein the concentration of silk fibroin in the aqueous solution is from about 5 mg/mL to about 100 mg/mL.

In certain embodiments, the invention relates to any of the methods described herein, wherein the concentration of API in the aqueous solution is from about 1 mg/mL to about 25 mg/mL. In certain embodiments, the API is a protein, and the concentration of API in the aqueous solution is from about 1 mg/mL to about 10 mg/mL.

In certain embodiments, the invention relates to any of the methods described herein, wherein annealing the silk/API droplets comprises exposing the silk/API droplets to methanol vapor.

In certain embodiments, the invention relates to any of the methods described herein, wherein the silk/API droplets are exposed to methanol vapor at a temperature from about 20° C. to about 24° C. for a time period of about 12 h to about 36 h.

In certain embodiments, the invention relates to any of the methods described herein, wherein annealing the silk/API droplets comprises heating, in the presence of a water bath, the silk/API droplets at a temperature from about 30° C. to about 70° C. at a pressure from about 10 mmHg to about 30 mmHg for a time period from about 6 h to about 36 h. In certain embodiments, the pressure is about 20 mmHg. In certain embodiments, the period of time is from about 12 h to about 24 h.

In certain embodiments, the invention relates to a method of treating cancer comprising administering by intratumoral injection to a cancerous tumor in a subject in need thereof a therapeutically effective amount of any of the pharmaceutical compositions described herein, provided the API is not an imaging agent.

In certain embodiments, the invention relates to a method of imaging a cancerous tumor comprising administering by intratumoral injection to the cancerous tumor in a subject in need thereof an effective amount of any of the pharmaceutical compositions described herein, provided the API comprises an imaging agent.

In certain embodiments, the invention relates to any of the methods described herein, wherein the cancerous tumor is breast cancer, pancreatic cancer, or prostate cancer.

### EXEMPLIFICATION

### Example 1: Fabrication of the Micro-Depots

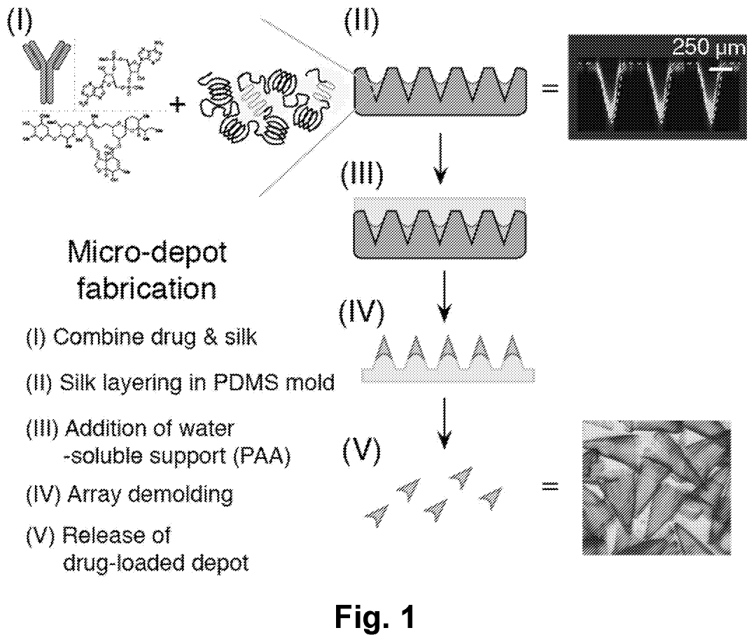

A molding process employed to fabricate the micro-depots described herein is illustrated in FIG. 1. Silk fibroin was prepared as an aqueous solution using silk worm cocoons according to the method described in Rockwood, D. N., Preda, R. C., Yucel, T., Wang, X., Lovett, M. L., and Kaplan, D. L. (2011) Materials fabrication from Bombyx mori silk fibroin, Nat Protoc 6, 1612-1631, incorporated herein by reference in its entirety.

The mixture was molded using a silicone array with micron-size pyramidal cavities (250 μm square base, 550 μm height). The molding process ensured high encapsulation of silk into the mold cavities. It involved filling the mold cavities with water, then adding the silk mixture as a droplet to each cavity (10-50 pL per cavity), as described in Vrdoljak, A., Allen, E. A., Ferrara, F., Temperton, N. J., Crean, A. M., and Moore, A. C. (2016) Induction of broad immunity by thermostabilised vaccines incorporated in dissolvable microneedles using novel fabrication methods, J Control Release 225, 192-204, incorporated herein by reference in its entirety.

To remove the drug-loaded matrix, the mold was filled with a water-soluble polyacrylic acid (PAA) polymer that filled the cavities and provided a pedestal to facilitate demolding of the silk tip, as described in DeMuth, P. C., Min, Y., Irvine, D. J., and Hammond, P. T. (2014) Implantable silk composite microneedles for programmable vaccine release kinetics and enhanced immunogenicity in transcutaneous immunization, Adv Healthc Mater 3, 47-58, incorporated herein by reference in its entirety. The silk micro-depots were then released by dissolving the water-soluble pedestal.

Aqueous solution of silk fibroin was combined with desired therapeutic (also referred to herein as active pharmaceutical ingredient (API)). Silk solutions ranged in concentration from 15 mg/mL to 60 mg/mL. Up to 5 μL of silk solution was loaded per mold (0.9 to 3.7 μg silk fibroin per tip).

In the experiments described in Examples 2 and 3, the API was an anti-CD73 blocking antibody available from BD Pharmingen™ as product no. 550738. The concentration of the antibody was 4-5 mg/mL when combined with silk fibroin. At 5 μL volume per mold, the achieved loading was up to 25 μg ant-CD73 per mold (0.3 μg per silk tip).

Additionally, the following APIs were successfully loaded into the silk micro-depots: Ovalbumin (a 45 kD protein, ovalbumin-dye conjugates available from Molecular Probes®), a soluble HIV-1 envelope trimer protein, a nucleic acid (polylC available from Invivogen®, Catalog # tlrl-pic, tlrl-pic-5), Cyclic diguanylate monophosphate (CDN, Invivogen®, Catalog # vac-nacdg), Synthetic triacylated lipoprotein Pam3CSK4 (Invivogen®, Catalog # vac-pms).

### Example 2: Methanol Annealing Improves Drug Loading

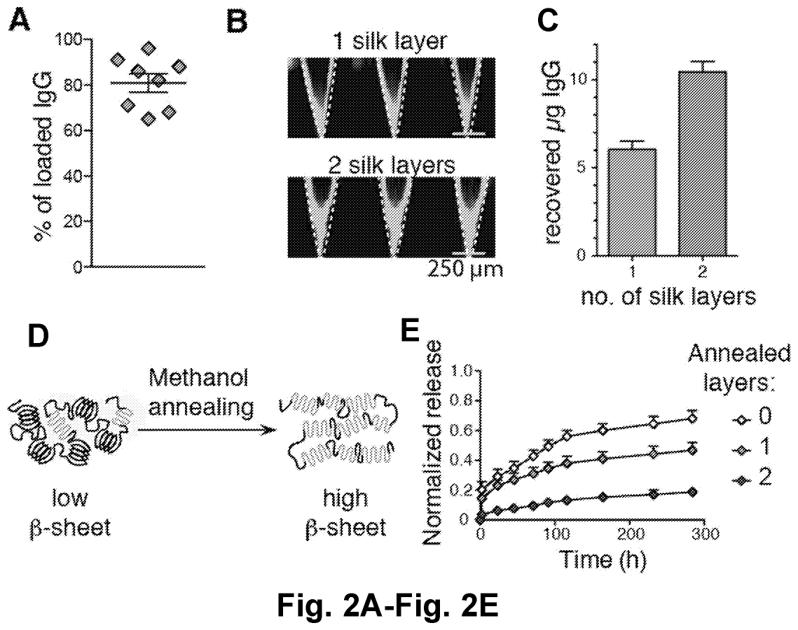

This procedure described in Example 1 was validated to yields high drug loading. The silk micro-depots were dislodged and degraded, allowing release of encapsulated anti-CD73 antibody. It was found that 80-90% of cargo loaded onto the silicone mold was released from the depots (FIG. 2A). To increase the amount of drug loaded, repeated addition of silk mixture to the silicone molds were used (FIG. 2B). Using this strategy, the number of layers of deposited silk matrix can be increased and nearly double the amount of drug can be loaded into each micro-depot (FIG. 2C).

Fabrication methods were employed that allow control of release kinetics from the silk micro-depots. Silk secondary structure is composed of repeating hydrophobic blocks that preferentially fold into anti-parallel β-sheets. Stacking of the β-sheets creates crystalline regions in the silk matrix that are resistant to protease degradation and matrix swelling. It was found that exposing molded silk to methanol vapor for a period of 18-24 hours results in silk with dramatically increased β-sheet content. Additionally, by treating molded silk with methanol vapor, silk tips having delayed release kinetics were generated. While silk depots with no annealing release 5% of encapsulated protein cargo per day, depots with 1 or 2 annealed layers released at 3% and 1% respectively (FIG. 2E). The results indicate that methanol annealing increases silk β-sheet content and results in slowed release of the API.

### Example 3: Injectable Formulations

Injectable formulations of the silk micro-depots were prepared as described below.

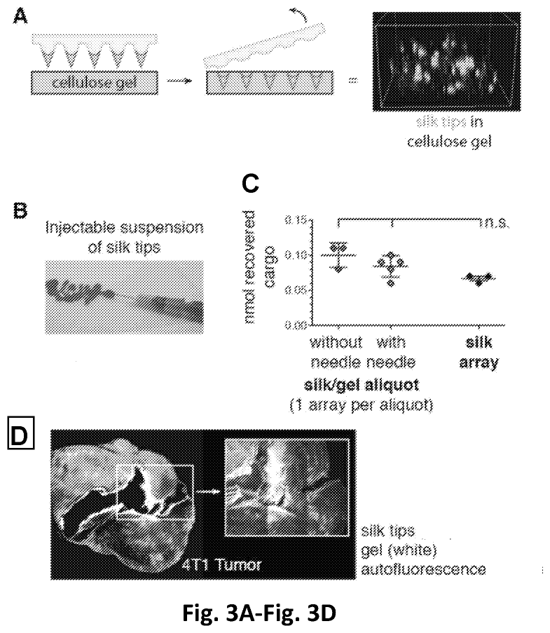

As a carrier for the depots, a carboxymethyl cellulose (CMC) gel was used. CMC is a viscosity promoter. At sufficient concentration in aqueous solution, CMC solutions form a gel with shear-thinning properties that allow passage through a syringe. Silk micro-depots were suspended by first embedding the array into CMC gel, then allowing PAA dissolution to release the tips from the remaining pedestal (FIG. 3A). The gel suspension was then loaded and dispensed with a syringe (FIG. 3B). The dispensed dose was determined by controlling the number of arrays loaded into a given volume of CMC gel. For example, where one array was loaded per 20 μL of CMC gel, and 20 μL aliquots were dispensed by syringe, it was determined that each aliquot contained a comparable dose of cargo to a single silk array (FIG. 3C). The density of loaded silk depots can thus be increased by suspending the tips in a smaller volume of CMC gel.

The injection of the silk micro-depots into diseased tissues was also investigated. The silk micro-depots were injected into 4T1 orthotopic mouse tumors and the whole tumors were imaged. It was found that silk micro-depots were suspended throughout the depth of the tumor (FIG. 3D).

### Example 4: Small Molecule as an API

This example demonstrates loading of small molecule, Alexa AF467® dye, available from ThermoFisher Scientific, in the silk micro-depots.

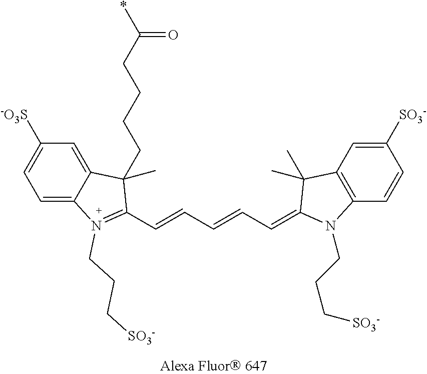

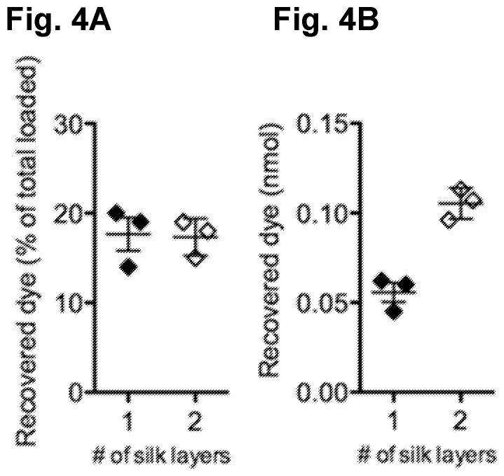

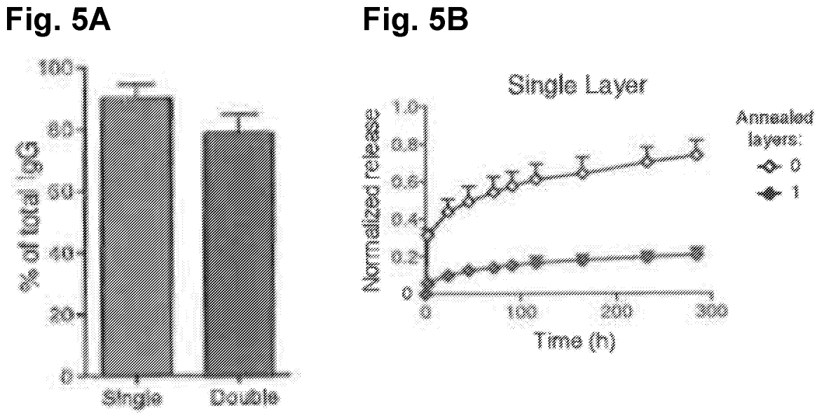

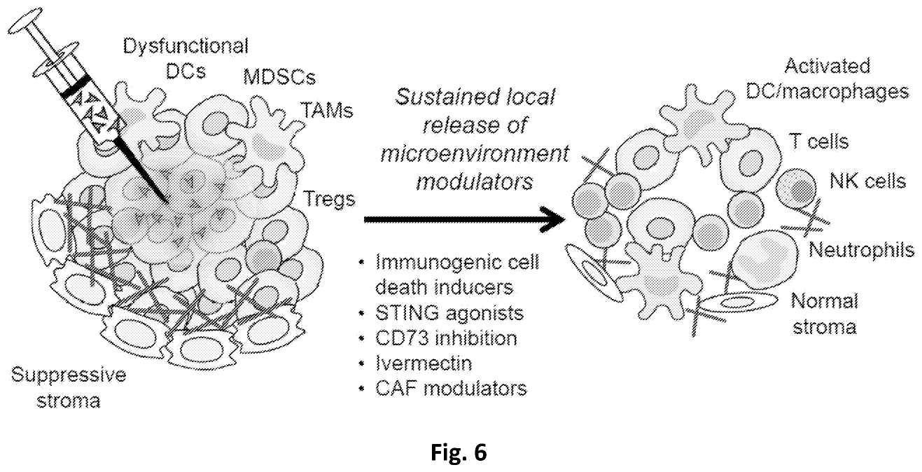

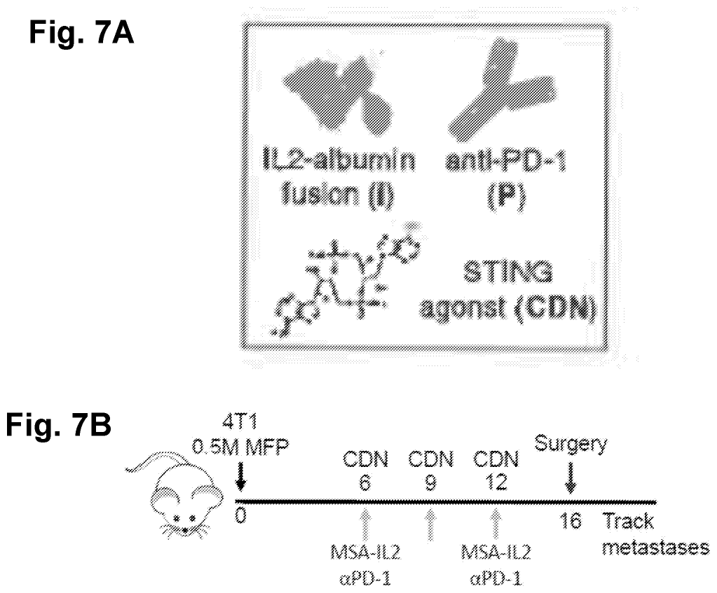

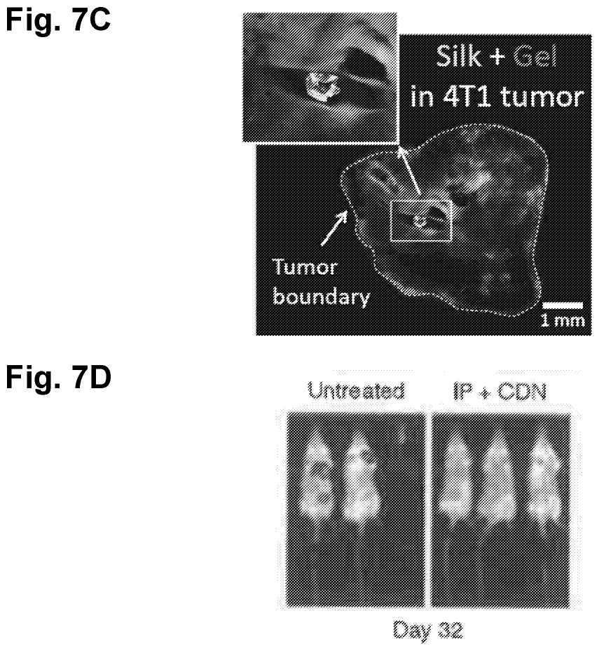

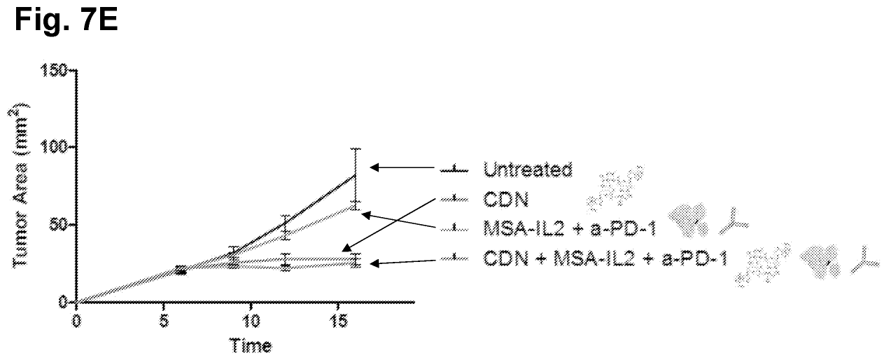

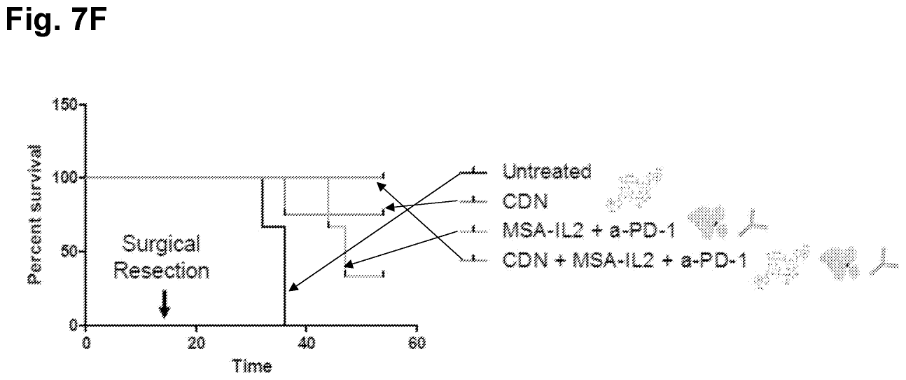

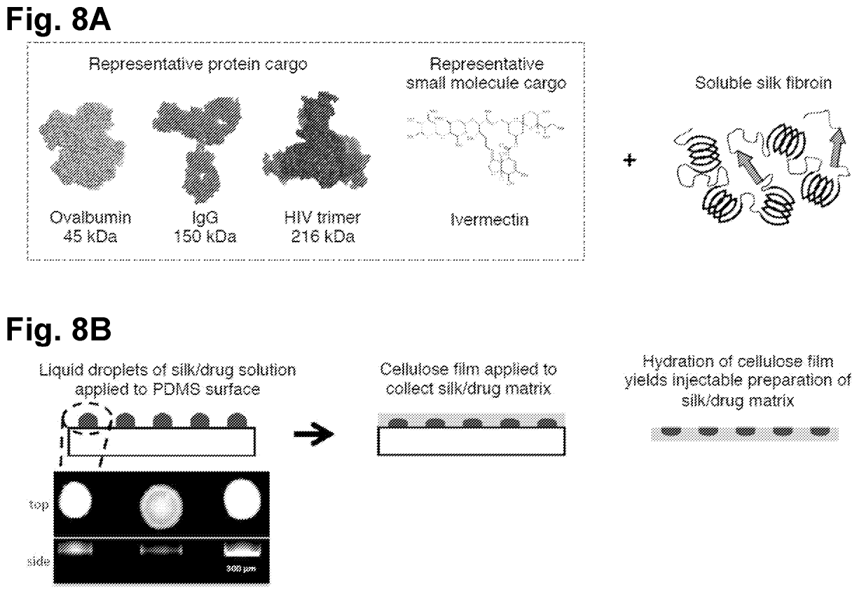

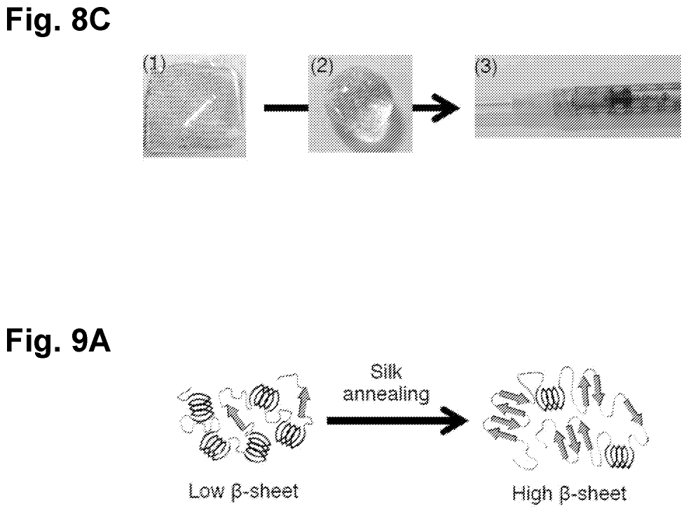

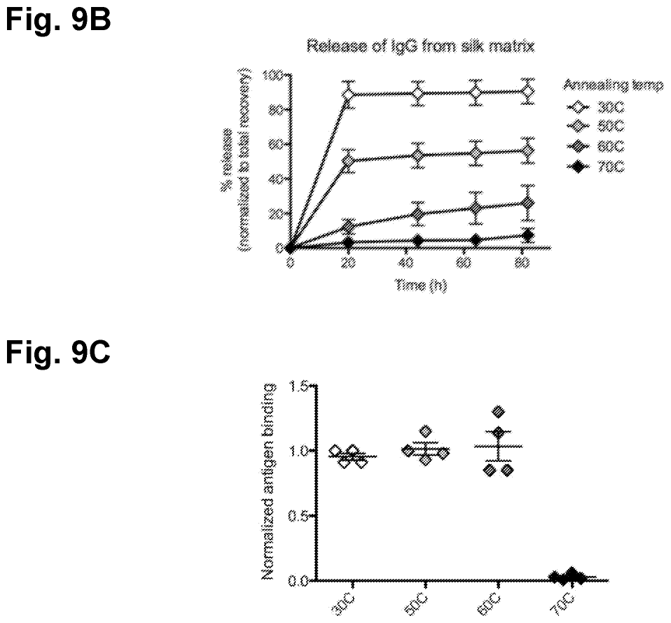

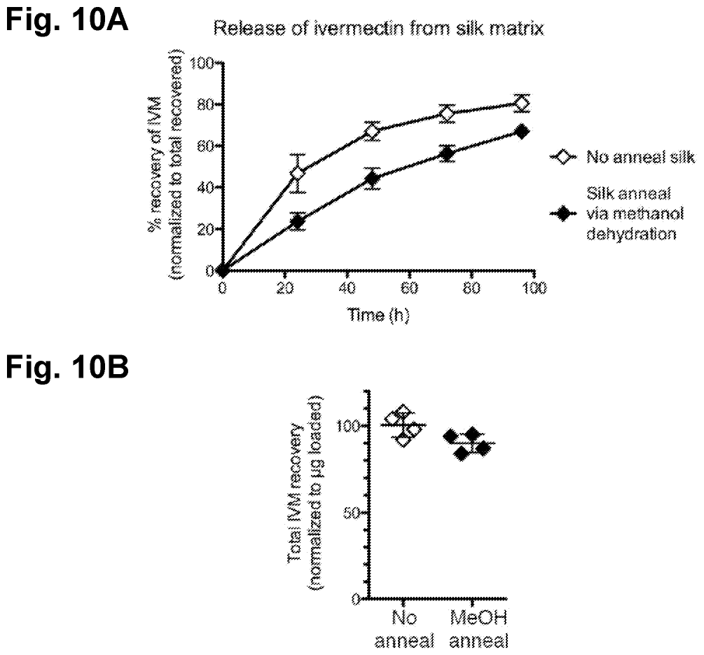

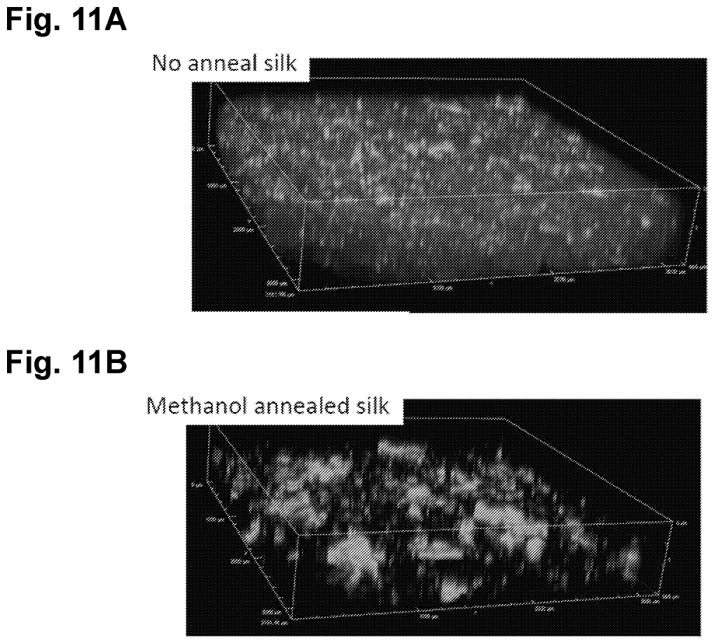

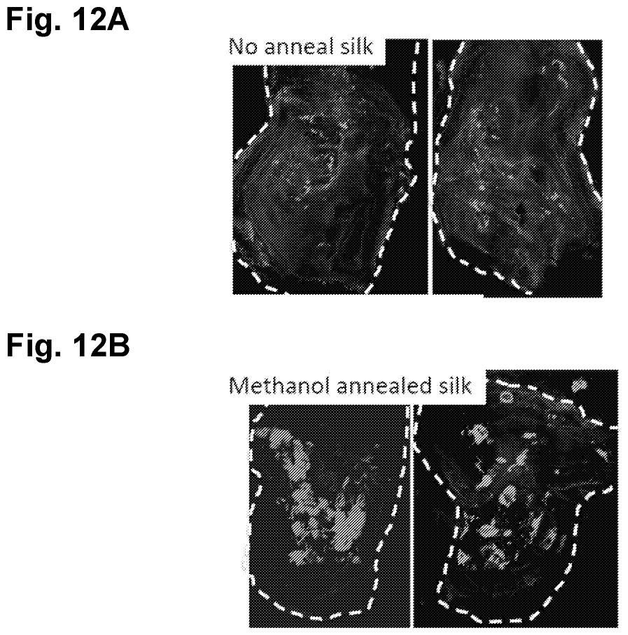

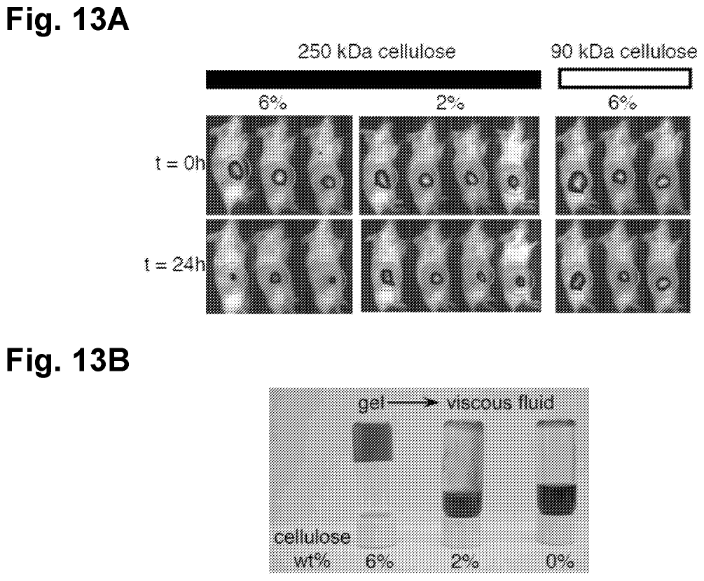

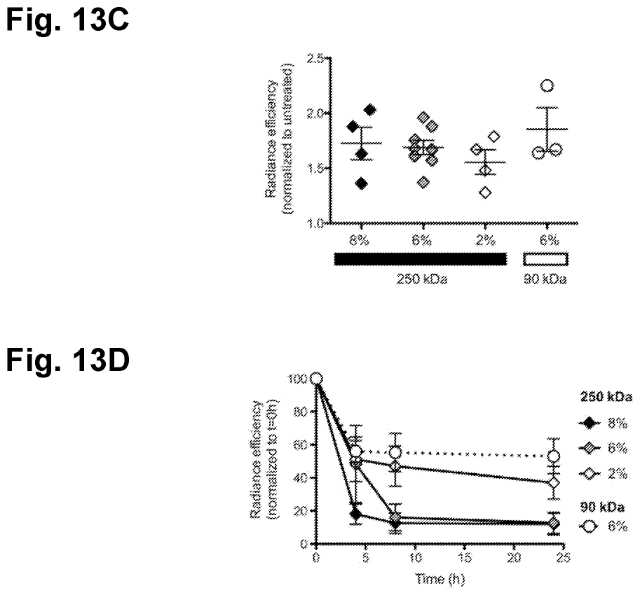

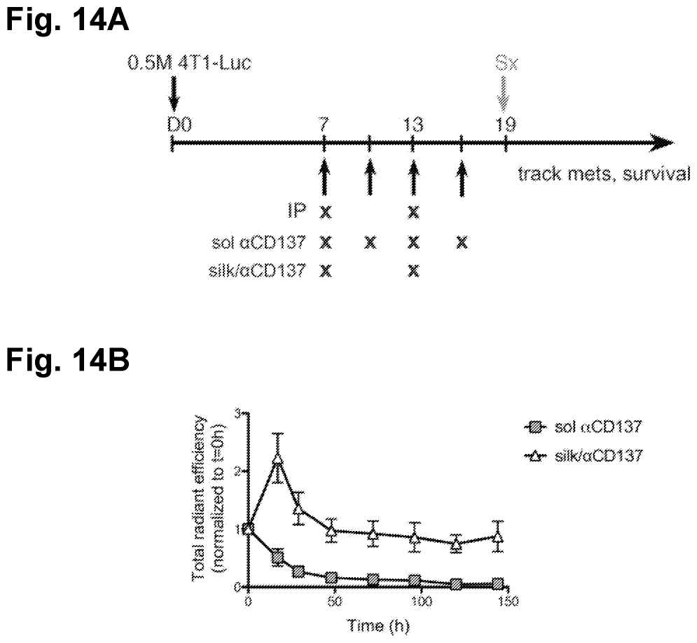

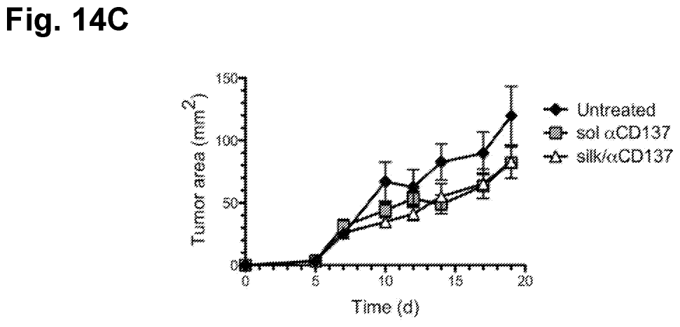

The silk was combined with the dye alexa fluor 647 (AF647) and molded to form the silk depots. The amount of dye deposited in the depots was quantified. FIG. 4A and FIG. 4B shows recovery as a percentage of the amount loaded (0.3 nmol per mold for single layer depots and 0.6 nmol for double layer depots) (FIG. 4A) and as total quantity in nmol (FIG. 4B).

### Example 5: Fabrication of Micro-Depots

Preparation of Silk Fibroin Solution

Cocoons were provided by Vaxess (Cambridge, Mass.). All other chemicals were purchased from Sigma (St. Louis, Mo.) and used as provided. Silk cocoons were cut into small pieces (approximately 1×1 cm) and boiled in a 0.02 M sodium carbonate solution for 40 minutes (1.25 g silk per 400 mL of solution). The silk fiber was rinsed extensively in deionized water and then dried. The silk was dissolved in a solution of 9.0 M lithium bromide at 60° C. for 4 hours (1.0 g silk per 4.0 mL lithium bromide solution). The lithium bromide salt was removed by extensive dialysis into deionized water using a Slide-a-Lyzer dialysis cassette (Pierce, Rockford, Ill.) for 24 hours. The resulting solution was centrifuged for 20 minutes at 10000×g to pellet impurities and the supernatant was collected. The concentration of the silk solution was determined by weighing residual solid obtained from a known volume or by measurement of absorbance at 280 nm. The silk fibroin solution was then diluted to 50 mg/mL using deonized water and stored at 4° C. The silk solution was filtered through a 0.45 μm syringe filter prior to use.

Preparation of Labeled Silk Fibroin

Silk fibroin was combined with a solution of 1.0 M sodium bicarbonate (pH 8.5) at a ratio of 10:1 (v/v). A solution of a succinimidyl ester alexa fluor 647 dye prepared at 10 mg/mL in anhydrous dimethyl sulfoxide was added (at a molar ratio of 2:1 dye to silk fibroin). mixture was incubated on a shaker for 2 hours. The solution was diluted 4-fold with PBS and purified by FPLC using a Superdex 200 Increase 10/300 G1 size-exclusion column (GE Healthcare). Fractions containing labeled silk were combined and concentrated using an amicon filter (3 kDa MWCO, Millipore Sigma). The concentration and degree of labeling were determined by absorption.

Preparation of API/Silk Mixture

A solution of silk fibroin solution was concentrated to 50-150 mg/mL using an amicon ultra centrifugal filter (3 kDa MWCO, Millipore Sigma) and final concentration was determined by absorption at 280 nm. A solution of API in PBS (2-15 mg/mL for protein API and 5-50 mg/mL for small molecule API) was combined with a solution of silk fibroin (50-150 mg/mL) to give the desired final concentration of silk fibroin (5-100 mg/mL) and API (1-10 mg/mL for protein API and 1-25 mg/mL for small molecule API).

Preparation of Molding Surface

Poly(dimethyl siloxane) (PDMS) microneedle molds (Sylgard 184, Dow-Corning, Midland, Mich.) were prepared using a laser micromachining instrument. The molds contained a 9×9 array of cavities with either pyramidal or concave shapes (cavity opening 200-400 μm, depth 200-600 μm). The molds were wetted with water immediately prior to silk application. A flat PDMS surface was prepared by curing PDMS mixture in 60 mm2 petri dishes. The surface was rinsed with acetone and dried prior to silk application.

Preparation of Molded Silk Matrix

A solution of silk/API was loaded into a 100 μL glass syringe (Hamilton Company, Reno, Nev.). The solution was applied as droplets onto either a wetted PDMS mold or a flat PDMS surface using a syringe pump (New Era Pump Systems, Inc, Farmingdale, N.Y.) at approximately 10-100 pL per droplet. For layering, the silk droplets were allowed to dry for 10-20 minutes and a second layer of silk/API solution was applied. The droplets were then dried under ambient conditions for 12-24 hours.

Annealing

Methanol annealing was performed by placing silk/API matrix molded onto PDMS into a sealed chamber containing a bath of methanol at 20-24° C. for 18-24 hours. The silk/API micro-depots were removed and dried for 12-24 hours. Percent beta-sheet crystallinity following methanol annealing is reported at 50-60% (Hu, X., et. al., Biomacromolecules, 2011, 12, 1686-1696)

Water annealing was performed by placing silk/API matrix molded onto PDMS into a vacuum oven containing a bath of water. The chamber was placed under vacuum (20 mmHg) and heated (30-70° C.) for 12-24 hours. The silk/API micro-depots were removed and dried for 12-24 hours. Percent beta-sheet crystallinity following water annealing depends upon temperature and is reported at 40-55% for annealing at 30-70° C. (Hu, X., et. al., Biomacromolecules, 2011, 12, 1686-1696).

Pedestal Formation

Polyacrylic acid (PAA) pedestal: a mixture of PAA (250 kDa, 35% w/v) was added to PDMS containing molded silk. The molds were centrifuged (10 min, 450×g) and dried at 20-24° C. (48 h on benchtop and 2-14 days under dessication). The PAA was demolded to remove the silk/API array from the PDMS mold.

Carboxymethyl cellulose (CMC) pedestal: a solution of CMC (0.2-2.0% w/v) was added to PDMS containing molded silk. The solution was dried on benchtop at 20-24° C. for 12-24 hours. The CMC film was demolded to remove the silk/API array from the PDMS mold. The resulting silk/API micro-depots are 100-600 μm in size.

### Example 6: Release of API from Silk/Microdepots In Vitro

Gel suspension containing silk-microdepots was aliquoted into spin columns (Thermo). A mixture of mouse serum in PBS (20% v/v) was added to the column (50-100 μL). The solution was incubated for 24 hours and drained by centrifuging the column into a collection tube (2000×g for 5 minutes). Fresh serum solution was replaced on a daily basis for the desired time course of the experiment (4-14 days). Remaining silk matrix was degraded by 12-24 hour treatment with a mixture of proteinase K (10 mg/mL) and protease XIV (10 mg/mL). The concentration of API was measured in collected supernatant using a plate reader (for fluorescent API) or by HPLC.

### Example 7: Injectable Formulations

PAA pedestal: silk/API matrix were sheared from PAA pedestal using sharp edge of a razor blade. Silk micro-depots were collected and mixed into a CMC mixture (4-10% w/v) using the plunger from a sterile syringe. Alternatively, the PAA pedestal was settled onto a CMC mixture (4-10% w/v) for a sufficient period to release the silk/API micro-depots into the CMC gel (5-10 minutes). The leftover PAA pedestal was removed and discarded. The CMC mixture containing silk/API micro-depots was backfilled into a 1 mL syringe prior to in vivo injection.

CMC Pedestal:

PBS was added to CMC film embedded with silk/API micro-depots to give a final CMC concentration of 2-10% w/v. The hydrated film was mixed using the plunger of a sterile spatula. The resulting gel suspension was loaded into a 1 mL syringe prior to in vivo injection.

The silk/API matrix were suspended in CMC at a final silk concentration of 3-15 mg/mL.

### Example 8: In Vivo Assessment of CMC Silk Microdepots

BALB/c mice were purchased from The Jackson Laboratory. Experiments were conducted using female mice, 7-10 weeks of age. Mice were inoculated with 0.5×106 4T1 cells (ATCC) via a subcutaneous injection in the mammary fat pad. Tumors were allowed to develop to 9-25 mm². Mice were placed under anesthesia using an isoflurane vapor chamber. A syringe loaded with a CMC gel suspension containing silk micro-depots was inserted into the tumor and 10-50 mL of gel suspension was injected using a 21-23 gauge needle.

Live whole animal imaging was performed using a Xenogen IVIS Spectrum (Caliper Life Sciences, Hopkinton, Mass.) on anesthetized mice. Fluorescence data was processed using region of interest (ROI) analysis with background subtraction using Living Image 4.0 software package (Caliper).

Tumors were excised following injection and fixed in 1% formaldehyde for 24 hours at 4° C. They were then embedded in a 3% (w/v) solution of low melting point agarose in PBS. The embedded tissues were sectioned using a vibratome VT1000 S (Leica Biosystems). The resulting sections were imaged by confocal microscopy.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

While this invention has been particularly shown and described with references to example embodiments thereof, it will be understood by those skilled in the art that various changes in form and details may be made therein without departing from the scope of the invention encompassed by the appended claims.

1. A particle comprising:
silk fibroin; and
at least one active pharmaceutical ingredient (API).

2. The particle of claim 1, wherein the silk fibroin is Bombyx mori silk fibroin.

3. The particle of claim 1, wherein the API is a protein, a nucleic acid, or a small molecule.

4. The particle of claim 1, wherein the API is selected from:
a STING agonist, a CD73 inhibitor, a CAF modulator, an immunomodulatory antibody, an adjuvant, a cytokine, and an imaging agent.

5. The particle of claim 1, wherein the API is selected from:
an anti-CD73 antibody, IgG, ovalbumin, a HIV-1 envelope trimer protein, polyIC, cyclic diguanylate monophosphate (CDN), Pam3CSK4, and ivermectin.

6. The particle of claim 1, wherein the particle has a characteristic size from about 50 μm to about 800 μm.

7. The particle of claim 1, wherein the API is suspended in the silk fibroin.

8. The particle of claim 1, wherein the amount of the API in the particle is from about 10 pg to about 0.6 μg.

9. The particle of claim 1, wherein the amount of silk fibroin in the particle is from about 50 pg to about 10 μg.

10. The particle of claim 1, wherein the particle is substantially conical in shape or substantially spherical.

11. (canceled)

12. A pharmaceutical composition, comprising:
a carboxymethyl cellulose (CMC) gel comprising CMC and a pharmaceutically acceptable carrier; and
a plurality of particles suspended within the CMC gel,
wherein at least one particle is a particle of claim 1.

13. (canceled)

14. The pharmaceutical composition of claim 12, wherein the molecular weight of the CMC is from about 50 kDa to about 500 kDa.

15-17. (canceled)

18. The pharmaceutical composition of claim 12, wherein the concentration of CMC in the CMC gel is from about 1% to about 8% by weight.

19-20. (canceled)

21. The pharmaceutical composition of claim 12, wherein the particles are present at a concentration from about 2 mg/mL to about 20 mg/mL in the CMC gel.

22. A method of manufacturing a particle of claim 1, comprising:
combining silk fibroin and at least one API in an aqueous solution;
placing the aqueous solution into a mold cavity, thereby coating a mold cavity with a silk/API layer;
placing a water-soluble polymer into the mold cavity, thereby coating the mold cavity with a silk/API/polymer layer;
removing the silk/API/polymer layer from the mold cavity; and
dissolving the polymer, thereby forming the particle.

23-24. (canceled)

25. The method of claim 22, further including annealing the silk/API layer.

26-28. (canceled)

29. The method of claim 22, further comprising placing a second amount of the aqueous solution into the mold cavity, thereby coating the silk/API layer with a second silk/API layer.

30. The method of claim 22, wherein the water-soluble polymer is selected from polyacrylic acid, gelatin, hydrolyzed gelatin, sodium polyacrylate, partially neutralized polyacrylic acid, polyacrylic acid-starch complexes, polyvinyl alcohol, polyvinylpyrrolidone, hydroxypropylcellulose, hydroxypropyl methylcellulose, hydroxyethyl cellulose, methylcellulose, carmellose sodium, carboxyvinyl polymer, methoxy ethylene-maleic anhydride copolymers, N-vinyl acetamide copolymers, xanthan gum, and gum arabic.

31. The method of claim 22, further including combining the particle with a carboxymethyl cellulose (CMC).

32. A method of manufacturing a pharmaceutical composition of claim 12, comprising:
combining silk fibroin and at least one API in an aqueous solution;
placing droplets of the aqueous solution onto a surface, thereby forming silk/API droplets;
annealing the silk/API droplets, thereby forming a plurality of particles;
coating the particles with CMC, thereby forming a silk/API-loaded film; and
hydrating the silk/API-loaded film, thereby forming the pharmaceutical composition.

33. The method of claim 32, further comprising removing the silk/API-loaded film from the surface prior to hydration.

34-39. (canceled)

40. A method of treating cancer comprising administering by intratumoral injection to a cancerous tumor in a subject in need thereof a therapeutically effective amount of a pharmaceutical composition of claim 12, provided the API is not an imaging agent.

41. A method of imaging a cancerous tumor comprising administering by intratumoral injection to the cancerous tumor in a subject in need thereof an effective amount of a pharmaceutical composition of claim 12, provided the API comprises an imaging agent.

42. (canceled)
